(19) United States
(12) Patent Application Publication
(10) Pub. No.: US 2023/0046617 A1
(43) Pub. Date: Feb. 16, 2023

(54) METHOD FOR PROMOTING EXPANSION OF HEMATOPOIETIC STEM CELLS AND AGENT FOR USE IN THE METHOD

(71) Applicant: FARON PHARMACEUTICALS OY, Turku (FI)

(72) Inventors: Sirpa JALKANEN, Piispanristi (FI); Imtiaz IFTAKHAR-E-KHUDA, Turku (FI)

(73) Assignee: FARON PHARMACEUTICALS OY, Turku (FI)

(21) Appl. No.: 17/789,252

(22) PCT Filed: Jan. 22, 2021
(86) PCT No.: PCT/FI2021/050039

§ 371 Date: Jun. 27, 2022

(30) Foreign Application Priority Data
Jan. 24, 2020 (FI) 20205073

(51) Int. Cl. C12N 5/0789; A61K 31/137; A61P 7/00

(52) U.S. Cl. CPC C12N 5/0647; A61K 31/137; A61P 7/00; C12N 2501/125; C12N 2501/145

(57) ABSTRACT

A vascular adhesion protein-1 (VAP-1) inhibitor can be used as a regulator of reactive oxygen species (ROS) concentration in ex vivo culturing of hematopoietic stem cells, which enables a method of producing an expanded population of hematopoietic5 stem cells ex vivo. Further, a VAP-1 inhibitor can be used in the treatment of bone marrow suppression or bone barrow failure in an individual.

### FIELD OF THE INVENTION

The present invention relates to a method for promoting expansion of hematopoietic stem cells and agent(s) suitable for use in expansion of hematopoietic stem cells.

### BACKGROUND OF THE INVENTION

Transplantation of hematopoietic stem cells (HSCs) collected from bone marrow (BM) or umbilical cord blood (CB) collected from healthy donors is used as a cure for several hematopoietic pathologies including e.g. leukemias, severe aplastic anemia, lymphomas, multiple myeloma and immune deficiency disorders. Thereby, the diseased hematopoietic cells including the HSCs are ablated and replaced by the healthy cells. Postnatal hematopoiesis and maintenance of hematopoietic stem cells mainly occur in the bone marrow, where HSCs and their progeny reside in specialized niches.

Hematopoietic stem cells (HSCs) are highly dependent on the perivascular stem cell niche in bone marrow (BM). Identification of the interactions between HSCs and their microenvironments may help to identify clinical approaches and opportunities in the field of hematopoietic stem cell transplantation and treatments affecting hematopoiesis. Therefore, a better understanding of the mechanisms that regulate hematopoiesis would aid understanding of hematological diseases and may also help in the development of new methods for ex vivo expansion of HSCs, since the number of HSCs that can be obtained for clinical transplantation from donors is limited, methods to promote expansion of HSCs are desirable.

### SUMMARY OF THE INVENTION

Now, it has been found that vascular adhesion protein-1 (VAP-1) is a component of the stem cell niche and plays a role in the maintenance and expansion of hematopoietic stem cells (HSCs). It has been found that VAP-1 is expressed by bone marrow vasculature in close proximity to hematopoietic stem cells and a lack of VAP-1 affects the number of HSCs and hematopoietic stem and progenitor cells (HSPCs) in the bone marrow (BM). It has been found that the inhibition of enzyme activity of VAP-1 facilitates expansion of umbilical cord blood and bone marrow derived HSCs.

In addition to the role of VAP-1 in the expansion of human HSCs, the inventtors of the present application also found a unique human VAP-1⁺HSC subpopulation. More specifically, it has been found that a subset of primitive human hematopoietic stem cells is VAP-1 positive and especially their expansion can be achieved by inhibiting the enzyme activity of vascular adhesion protein-1 (VAP-1).

The findings of the present invention provide a method for expanding HSCs in clinical applications using VAP-1 inhibitors. The findings of the present invention may help to improve bone marrow recovery after injury, enhance the effects of bone marrow transplantation and ameliorate the mobilization, harvesting and expansion of HSCs. Further, the findings of the present invention provide a novel method for treating several hematological diseases or conditions, which benefit from expanded population of hematopoietic stem cells. The present invention provides a method for treating a condition in which bone marrow does not function normally and the patient is in need of boosting his/her hematopoiesis. In one aspect, the findings of the present invention provide a novel efficient method for increasing ex vivo the number of umbilical cord blood HSCs, since umbilical cord blood transplantation (UCBT) has become an established therapy for patients without matched donors, leading to cures of previously incurable disease.

Vascular adhesion protein-1 (VAP-1) is a transmembrane protein also known as copper-containing amine oxidase (AOC 3) or semicarbazide-sensitive amine oxidase (SSAO). The extracellular amine oxidase activity of VAP-1 catalyzes oxidative deamination of primary amines. The reaction results in the formation of the corresponding aldehyde and release of ammonia and H₂O₂, one of the reactive oxygen species (ROS). According to the present invention, it has been observed that a VAP-1 inhibitor reduces SSAO-specific hydrogen peroxide generation. More detailed, in the present invention it has been found that a VAP-1 inhibitor can be used to maintain consistent level of the reactive oxygen species (ROS) needed and thereby promoting an expansion of the HSCs. The maintenance, expansion and differentiation of HSCs are extremely sensitive to the ROS concentrations. The present invention provides a method for controlling the ROS concentration by inhibiting the enzymatic activity of VAP-1 using a VAP-1 inhibitor, wherein a level of ROS is reduced to a level providing growth advantage to HSCs. In the present invention, a VAP-1 inhibitor which blocks or inhibits the enzyme activity of VAP-1, more specifically amine oxidase activity of VAP-1, is used to influence the concentration of ROS. The present invention is based on the improved expansion of HSCs using inhibitor compounds that influence the concentration of ROS.

According to one aspect of the present invention, a VAP-1 inhibitor, also called as SSAO inhibitor, capable of inhibiting the enzymatic activity of vascular adhesion protein 1 (VAP-1) is used as a regulator of reactive oxygen species (ROS) concentration in ex vivo culturing of hematopoietic stem cells.

According to another aspect, the present invention provides a method of producing an expanded population of hematopoietic stem cells ex vivo, said method comprising culturing ex vivo hematopoietic stem cells with a vascular adhesion protein-1 (VAP-1) inhibitor capable of inhibiting the enzymatic activity of vascular adhesion protein 1 (VAP-1), wherein the VAP-1 inhibitor is present in an amount that is sufficient to produce an expanded population of hematopoietic stem cells. The present invention provides an improved method for ex vivo expansion of umbilical cord blood and bone marrow derived HSCs for transplantation.

Further, the present invention provides a cell expansion culture medium for hematopoietic stem cells comprising a vascular adhesion protein (VAP-1) inhibitor capable of inhibiting the enzymatic activity of vascular adhesion protein 1 (VAP-1).

According to a third aspect, the present invention also provides a method for promoting expansion of hematopoietic stem cells in an individual, comprising administering a VAP-1 inhibitor capable of inhibiting the enzymatic activity of vascular adhesion protein 1 (VAP-1) or a composition comprising a VAP-1 inhibitor capable of inhibiting the enzymatic activity of vascular adhesion protein 1 (VAP-1) to an individual. According to the present invention, a method of treating a disease or a condition that benefits from expanded population of hematopoietic stem cells, comprising administering a VAP-1 inhibitor capable of inhibiting the enzymatic activity of vascular adhesion protein 1 (VAP-1) to an individual suffering such disease or condition in an amount sufficient to produce expanded population of hematopoietic stem cells. According to an embodiment of the present invention, a VAP-1 inhibitor may be used in the treatment of bone marrow suppression or bone marrow failure, which refer to conditions in which bone marrow does not function normally and there is a need for the treatment affecting the number of HSCs.

### BRIEF DESCRIPTION OF THE DRAWINGS

FIG. 1. A schematic diagram of the role of ROS concentration in HSCs expansion. VAP-1/SSAO produces hydrogen peroxide (a species of ROS), ammonia, and aldehyde that is blocked by the inhibitor according to the present invention leading to the expansion of HSCs.

FIG. 2. VAP-1 is expressed on vascular endothelium and primitive HSCs in human BM and inhibition of VAP-1 increases the engraftment potential in NBSGW mice and the number of HSCs in CFU assays.

(A) Expression of VAP-1 in human bone marrow (BM). Tissue sections were stained with a polyclonal anti-VAP-1 antibody or rabbit IgG as a control. All observed blood vessels expressed VAP-1. Arrowheads indicate VAP-1-expressing arterioles, and arrows indicate venules. Scale bars 50 μm, (n=2).

(B) Flow cytometric identification of primitive HSCs in human BM. BM cells were stained with Lineage cocktail, anti-CD34, anti-CD38, anti-CD90, anti-CD45RA, anti-CD49f antibodies. The plots show the gating strategy for HSCs. Gates P-2, P-3, P-4, and P-5 show the sequential enrichment of HSCs, with gate P-5 representing the purest population.

(C) Expression of VAP-1 was analyzed in cells from gate P-5 (Lin⁻CD34⁺CD38⁻CD45RA⁻CD90⁺CD49f⁺) using anti-VAP-1 antibody JG-2; 19.5% of P-5 cells express VAP-1 (Data of one representative donor out of 4 is shown).

(D) Batch sorting of VAP-1⁻ and VAP-1^{+/lo} HSCs from fresh frozen human BM in the CD34⁺ gate. The frequency of VAP-1⁻ and VAP-1^{+/lo} subsets represents relative size of two subsets within the dot plot.

(E) In vivo engraftment of 19000 VAP-1⁻ or VAP-1⁻+VAP-1⁺ (16250 VAP-1−+2750 VAP-1⁺) FACS sorted human BM cells in non-irradiated NBSGW mice. Half of animal from each group were treated LJP-1586 (inhibitor) as described in experimental part. Six weeks after the transplantation the mice were sacrificed; BM were harvested and analyzed by flow cytometry. Representative flow cytometric plots from each group showing human CD45+ cells engraftment (percentage) in BM of the recipient mice.

(F) Summary of the percentages of human CD45⁺ cells engraftment in the BM of NBSGW mice. All four groups (VAP-1⁻ inhibitor or control treated and VAP-1⁻+VAP-1⁺ inhibitor or control treated) contain three animals each and equal number of BM cells as well as long term HSCs (CD90⁺CD49f⁺) were transplanted. The cut-off value for engraftment was set as 0.1%. The number of donor cells in BM at the end of the experiment are indicated.

(G) VAP-1 inhibition increases the number of HSCs in CFU assays. Five hundred human BM-derived CD34⁺ cells were cultured under CFU conditions in the presence of LJP-1586 (0.5 μM) or vehicle. After 12 days, cells were resuspended, replated a second time after increasing the volume of the culture by 10-fold, resuspended again, and replated a third time after increasing the volume of the culture by 5-fold. The results were calculated using cells derived from two donors made in triplicates. Student t-test was applied.

FIG. 3. Primitive HSCs in human umbilical cord blood (CB) express VAP-1. Expression of VAP-1 in CB cells. CD34⁺ cells were isolated from CB and stained for flow cytometry. Expression of VAP-1 was analyzed in cells from gate P-5. CB samples from ten donors were analyzed with anti-VAP-1 antibody JG-2. Data of one representative donor out of 10 is shown.

FIG. 4. LJP-1586 treatment facilitates expansion of umbilical cord blood (CB) derived HSCs in ex vivo.

(A) Effect of LJP-1586 on CD38⁻CD34⁺ cells. FACS sorted CD38⁻CD34⁺CB-derived cells were obtained from three donors (CB-1, CB-2, CB-3) and cultured in StemSpan SFEM medium II containing 1 μM LJP-1586 for 15 days (n=3).

(B) The cells shown in B were further analyzed for primitive HSCs using the additional criteria of CD45RA⁻CD90⁺CD49f⁺ expression as shown in gate P-4. Fold expansion subsequent to LJP-1586 treatment was calculated from the average of the three donors and is shown in the columns (n=3). Student t-test was applied.

(C) Long term effects of LJP-1586. One hundred human CB-derived Lin-CD38⁻CD34⁺VAP-1⁺ and VAP-1⁻HSCs were cultured in liquid conditions in presence of LJP-1586 (1 μM) or vehicle. After 10, 15 and 20 days, the cells were analysed for CD38⁻CD34⁺CD45RA⁻CD90⁺ expression as shown in FIG. 4B and C (gate P-3). Data are presented as percentages from the starting parent cells (CD38⁻CD34⁺ cells). Fold expansion of HSCs was calculated from the average of the ten donors. Student's t-test was applied.

(D) Effects analysed as CFUs. CB-derived cells obtained from the three donors were expanded in the presence or absence of LJP-1586 (0.5 μM) for 15 days in liquid culture and then analyzed by the CFU assay in the presence or absence of UP-1586. P-values were calculated using student's t-test.

FIG. 5. LJP-1586 reduces ROS production of HSCs in liquid cultures. ROS were detected by DHR-123 using living HSCs from 9-day liquid cultures containing 0.25M or 0.5M LJP-1586 respectively and analyzed by flow cytometry. Shown is the CD38−, CD34+ gated cells after activating them by PMA. Red DHR-123 turns green when oxidized. Closed histograms show control conditions, open histograms represent HSCs cultured in presence of LJP-1586. Cells are from one donor and two technical repeats.

FIG. 6. Structure of VAP-1 inhibitor szTU73 and its capacity to inhibit the enzymatic activity of VAP-1 in Amplex-Red assays.

FIG. 7. VAP-1 inhibitor szTU73 expands hematopoietic stem cells (CD34+CD38−CD90+CD45RA−). CD34+ cord blood-derived cells were cultured with different concentrations of szTU73 for 21 days. A: Flow cytometric analyses of 7-AAD− cells (live) using CD38 and CD34 as markers. B: Further analyses of 7-AAD−CD34+CD38− cells using CD90 and CD45RA as markers. Percentages of the positive cells within the gates are shown.

### DETAILED DESCRIPTION OF THE INVENTION

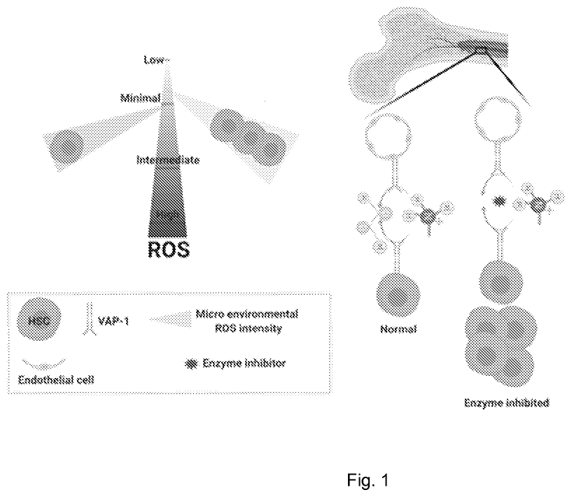

Vascular adhesion protein-1 (VAP-1) belongs to the family of copper-containing amine oxidase/semicarbazide-sensitive amine oxidases that catalyze the oxidative deamination of primary amines with subsequent production of aldehyde, ammonium and hydrogen peroxide (a species of ROS). FIG. 1 shows a schematic diagram of the role of ROS concentration in HSCs expansion and the function of the VAP-1 inhibitor according to the present invention in an expansion of HSCs. The amine oxidase activity of VAP-1 catalyzes oxidative deamination of amines into their corresponding aldehydes and produces ammonia and hydrogen peroxide. Hydrogen peroxide is one of the reactive oxygen species (ROS). The maintenance, expansion and differentiation of HSCs are extremely sensitive to the ROS concentrations. The enzymatic activity of VAP-1 leads to production of ROS, which influence the development and self-renewal of HSCs. Low levels of ROS are required for maintenance of HSCs and intermediate levels of ROS drive proliferation and differentiation, while high levels of ROS lead to damage and exhaustion of the stem cell pool. As the enzymatic activity of VAP-1 is not the sole source of ROS, VAP-1 inhibition can be used to fine-tune the ROS concentration. In the present invention, it has been found that a VAP-1 inhibitor can be used to maintain and control consistent level of ROS needed for promoting an expansion of the HSCs. According to the present invention, the enzymatic activity of VAP-1 is inhibited or reduced using a VAP-1 inhibitor, wherein a level of ROS is reduced to a level providing growth advantage to HSCs.

In the present invention, a VAP-1 inhibitor which blocks or at least inhibit the enzymatic activity of VAP-1, more specifically amine oxidase activity of VAP-1, is used to influence the concentration of ROS. According to one aspect of the present invention, a VAP-1 inhibitor, also called as SSAO inhibitor, is used as a regulator of reactive oxygen species (ROS) concentration in ex vivo culturing of hematopoietic stem cells and hence a VAP-1 inhibitor capable of inhibiting the enzymatic activity of vascular adhesion protein 1 (VAP-1) is used in promoting an expansion of HSCs in ex vivo culturing. After ex vivo culturing the expanded population of HSCs can be used in transplantation into an individual.

A method according to an embodiment of the present invention for producing an expanded population of hematopoietic stem cells ex vivo comprising culturing ex vivo a population of hematopoietic stem cells (HSCs) with a vascular adhesion protein 1 (VAP-1) inhibitor capable of inhibiting the enzymatic activity of vascular adhesion protein 1 (VAP-1), wherein the VAP-1 inhibitor is present in an amount that is sufficient to produce an expanded population of hematopoietic stem cells. A population of HSCs refers to a group including HSCs, i.e. the number of HSCs can be increased by the method according to the present invention.

HSCs can be cultured any suitable medium for the purpose and using known methods in the fields. A cell expansion culture medium according to the present invention for hematopoietic stem cells comprises a VAP-1 inhibitor. A concentration of a VAP-1 inhibitor in a culture medium depends on the inhibitor compound used. According to the present invention the VAP-1 inhibitor is present in an amount that is sufficient to produce an expanded population of hematopoietic stem cells. Lower or higher levels of the current inhibitor may lead less efficient expansion of HSCs. In an embodiment, the VAP-1 inhibitor can also be used to maintain the population of hematopoietic stem cells in ex vivo cultures. The degree of the HSC expansion is also donor dependent.

According to the present invention, said hematopoietic stem cells are human cells and derived from umbilical cord blood, bone marrow and/or peripheral blood. In a preferred embodiment, the present invention is used to expansion of umbilical cord blood and/or bone marrow derived HSCs in ex vivo cultures.

In an embodiment, the present invention provides an improved method for promoting expansion of HSCs originating from umbilical cord blood (CB). Umbilical CB can be used as a source of HSCs and although initially only used to treat children, its efficacy in adults has been increased by improvement of cell dosing and antigen matching. Unlike adult bone marrow (BM) donors, who can often donate multiple times for repeated transplantations, MHC matched umbilical CB is unique. Therefore, it would be helpful to expand and maintain umbilical CB-derived HSCs ex vivo according to a method of the present invention. Another problem associated with CB transplantation is delayed engraftment of immature HSCs and consequently a lack of rapidly proliferating multipotent progenitors. Inhibition of the enzymatic activity of VAP-1 may also overcome this problem.

According to the present invention, VAP-1/SSAO inhibitors that modulate VAP-1 enzymatic activity, more specifically amine oxidase activity of VAP-1, would be useful for the treatment of a disease or a condition that benefits from expanded population of hematopoietic stem cells, comprising administering a VAP-1 inhibitor or a compound comprising a VAP-1 inhibitor to an individual suffering such disease or condition. The present invention based on a method which promotes expansion of hematopoietic stem cells in an individual, comprising administering a VAP-1 inhibitor capable of inhibiting the enzymatic activity of vascular adhesion protein 1 (VAP-1) or a compound comprising said VAP-1 inhibitor to an individual.

According to the present invention, a VAP-1 inhibitor capable of inhibiting the enzymatic activity of vascular adhesion protein 1 (VAP-1) or a compound comprising said VAP-1 inhibitor is used in the treatment of a disease or a condition that benefits from expanded population of hematopoietic stem cells. According to an embodiment of the present invention, a VAP-1 inhibitor capable of inhibiting the enzymatic activity of vascular adhesion protein 1 (VAP-1) or a compound comprising said VAP-1 inhibitor is used in the treatment of bone marrow suppression or bone marrow failure, which refer in the present disclosure to a condition in which bone marrow does not function normally and there is a need for the treatment affecting the number of HSCs and the boosting of hematopoiesis.

Bone marrow failure or bone marrow suppression can be in association with multiple other diseases or conditions, such as leukemia, multiple myeloma, aplastic anemia, mentioned as an example. Bone marrow suppression, also referred to as myelosuppression is a condition in which bone marrow activity is decreased, resulting in fewer red blood cells, white blood cells and platelets. Because the bone marrow is the manufacturing center of blood cells, the suppression of bone marrow activity causes a deficiency of blood cells. This condition can rapidly lead to life-threatening infection, as the body cannot produce leukocytes in response to invading bacteria and viruses, as well as leading to anaemia due to a lack of red blood cells and spontaneous severe bleeding due to deficiency of platelets. Commonly, bone marrow suppression is e.g. a serious side effect of chemotherapy and/or certain drugs affecting the immune system. According to the present invention, a VAP-1 inhibitor(s) can be used in the treatment of bone marrow suppression by improving an expansion of HSCs and thereby boosting hematopoiesis. Also, in bone marrow failure an insufficient amount of red blood cells, white blood cells or platelets are produced. Bone marrow failure can be inherited or acquired after birth. According to the present invention, bone marrow failure or bone marrow suppression can be treated administering a VAP-1 inhibitor capable of inhibiting the enzymatic activity of vascular adhesion protein 1 (VAP-1) or a compound comprising a VAP-1 inhibitor to a patient, and/or with stem cells transplant, wherein a method according to the present invention for improved ex vivo culturing is advantageous.

According to an embodiment of the invention, a method for treating diseases or conditions that benefits from expanded population of hematopoietic stem cells, such as bone marrow suppression or bone marrow failure, comprises administering to an individual of therapeutically effective amounts of a VAP-1 inhibitor or a pharmaceutical composition comprising a VAP-1 inhibitor. The term “treatment” or “treating” shall be understood to include complete curing of a disease or disorder, as well as amelioration or alleviation of said disease or disorder. The term “therapeutically effective amount” is meant to include any amount of a VAP-1 inhibitor according to the present invention that is sufficient to inhibit enzyme activity of VAP-1 and produce expanded population of hematopoietic stem cells. Therapeutically effective amount may comprise single or multiple doses of VAP-1 inhibitor. The dose(s) chosen should be sufficient on inhibition of VAP-1 enzymatic activity and to promote an expansion of HSCs in an individual.

Administering refers to the physical introduction of a VAP-1 inhibitor or a pharmaceutical composition comprising a VAP-1 inhibitor to an individual, using any of the various methods and delivery systems known to those skilled in the art. According to the present invention, a VAP-1 inhibitor or a composition comprising a VAP-1 inhibitor may be administered by any means that achieve their intended purpose. According to an embodiment of the present invention, a VAP-1 inhibitor or a composition comprising a VAP-1 inhibitor may be administered orally and/or as an infusion. For example, administration may be intravenous, intramuscular, intraperitoneal, subcutaneous or other parenteral routes of administration, for example by injection or infusion therapy. In addition to the pharmacologically active compounds, the pharmaceutical compositions contain suitable pharmaceutically acceptable carriers comprising excipients and auxiliaries that facilitate processing of the active compounds into preparations that can be used pharmaceutically. According to the present invention, a VAP-1 inhibitor may be any suitable compound that inhibiting, affecting and/or modulating an enzymatic activity of VAP-1. In an embodiment of the present invention, a VAP-1 inhibitor comprises an inhibitor compound which is capable of inhibiting the enzymatic activity of vascular adhesion protein-1 (VAP-1), more specifically an inhibitor compound which is capable of inhibiting amine oxidase activity of VAP-1. According to an embodiment of the present invention, inhibitors of copper-containing amine oxidases, commonly known as semicarbazide-sensitive amine oxidases (SSAO), can be used as VAP-1 inhibitors, i.e. a VAP-1 inhibitor is also called as semicarbazide-sensitive amine oxidase (SSAO) inhibitor. SSAOs are enzymes that catalyze oxidative deamination of primary amines. According to an embodiment of the present invention the VAP-1/SSAO inhibitor is used to inhibit the activity of SSAO. According to an embodiment of the present invention, VAP-1/SSAO inhibitor can inhibit the SSAO activity of soluble SSAO or the SSAO activity of membrane-bound VAP-1.

According to an embodiment of the invention, a VAP-1 inhibitor comprises semicarbazide and/or hydroxylamine. According to an embodiment of the invention, semicarbazide and/or hydroxylamine can be used in ex vivo expansion method of HSCs.

According to an embodiment of the present invention, a VAP-1 inhibitor comprises antibodies or fragment(s) thereof and/or small molecule enzyme inhibitors that are capable of inhibiting the enzymatic activity of VAP-1. In an embodiment of the present invention, VAP-1 inhibitor comprises a small molecule inhibitor of VAP-1. Commonly, small molecule inhibitor refers to organic compound with a low molecular weight. According to an embodiment of the present invention a VAP-1 inhibitor may be any small molecule inhibitor which is capable of blocking and/or inhibiting the enzymatic activity of VAP-1, more detailed amine oxidase activity of VAP-1 and thereby reducing a level of ROS to a level providing growth advantage to HSCs. In an embodiment of the present invention, a VAP-1 inhibitor comprises a small molecule inhibitor of VAP-1 and/or a small molecule inhibitor of VAP-1 conjugated to a peptide capable of binding to VAP-1.

Many small molecule inhibitors have been developed or are under the development against VAP-1. According to an embodiment of the present invention, a VAP-1 inhibitor may be small molecule inhibitor, such as SSAO/VAP-1 inhibitor BI 1467335 (formerly known as PXS-4728A (4-(E)-2-(aminomethyl)-3-fluoroprop-2-enoxy)-N-tert-butylbenzamide)), PXS-4681A ((Z)-4-(2-(aminomethyl)-3-fluoroallyloxy)benzenesulfonamide hydrochloride), LJP-1586, PXS-4159, PXS-4206, TERN-201, ASP8232, SZV-1287 (3-(3,4-diphenyl-1,3-oxazol-2-yl)propanal oxime), UD-014, PRX167700, LJP 1207 (N′-(2-phenyl-allyl)hydrazine hydrochloride), szTU73 and/or RTU-009. These above-mentioned small molecular inhibitors are exemplary embodiments of VAP-1 inhibitors known in the market currently. These small molecule inhibitors are mentioned as non-restrictive examples only.

In an exemplary embodiment of the present invention, a VAP-1 inhibitor comprises Z-3-fluoro-2-(4-methoxybenzyl)allylamine hydrochloride (LJP 1586). LJP-1586 (Z-3-fluoro-2-(4-methoxybenzyl) allylamine hydrochloride) is an inhibitor that blocks the enzymatic activity of VAP-1 but does not affect its adhesive property. The compound is described for example in O'Rourke et al., “Anti-inflammatory effects of LJP-1586 [Z-3-fluoro-2-(4-methoxybenzyl)allylamine hydrochloride], an amine-based inhibitor of semicarbazide-sensitive amine oxidase activity”, Journal of Pharmacology and Experimental Therapeutics, February 2008, 324 (2), pp. 867-875.

### Experimental Section

Method Details

Immunohistochemistry

To visualize the VAP-1 expression in BM, anonymous human bone samples obtained from Turku University Hospital with the permission of its ethical authorities were decalcified, embedded in paraffin, and cut into 5 μm thick sections. Sections were de-paraffinized with xylene, rehydrated in a series of decreasing concentrations of ethanol, and treated with 10 mM sodium citrate (pH 6.0) for 10 min at 98° C. for antigen retrieval. To block endogenous peroxidase activity, sections were incubated in 1% H₂O₂ prepared in phosphate-buffered saline (PBS) for 30 min. Immunohistochemical staining with a polyclonal antibody against VAP-1 (1:500) and control rabbit IgG was performed at 4° C. overnight in accordance with the instructions provided with the VECTASTAIN ABC kit (Vector Laboratories). Samples were counter-stained with hematoxylin. Images were acquired using an Olympus BX60 microscope. Background subtraction and adjustment of brightness and contrast were performed using ImageJ software.

Bone Marrow Transplantations

Human fresh frozen BM CD34+ cells (LONZA) were thawed and stained with APC conjugated mouse anti-Lineage cocktail, PE-Cy7-conjugated anti-CD34 and FITC-conjugated monoclonal antibodies 1B2, TK8-14, and JG-2 against different epitopes of human VAP-1. For batch cell sorting of VAP-1^{+/lo} and VAP-1⁻ cells we used a Sony SH800 cell sorter with class A2 Level II biosafety cabinet using 130 μm microfluidic sorting chips. The NBSGW (immune-deficient, c-Kit-deficient) mice not needing irradiation to accept human cells were used as BM donors. In the VAP-1⁻ group 19000 cells and in the VAP-1−+VAP-1^{+/lo} group 16250 VAP-1⁻ cells and 2750 VAP-1^{+/lo} cells were intravenously injected per animal. One day after transplantation mice were intraperitoneally injected with VAP-1 inhibitor, LJP-1586 (O'Rourke et al., “Anti-inflammatory effects of LJP-1586 [Z-3-fluoro-2-(4-methoxybenzyl)allylamine hydrochloride], an amine-based inhibitor of semicarbazide-sensitive amine oxidase activity”, Journal of Pharmacology and Experimental Therapeutics, February 2008, 324 (2), pp. 867-875) at a dose of 10 mg/kg or with 100 μl of PBS as a control three times in a week for total of six weeks. At the end of the treatment the mice were sacrificed and BM were collected. BM cells were stained for anti-mouse CD45, anti-human CD45, anti-human CD34, anti-human CD19 together with anti-human CD33. Samples were run on LSR fortessa and the data was analyzed with FlowJo. Percentage of chimerism [% chimerism=(% test donor-derived cells)×100/((% test donor-derived cells+(% competitor-derived cells))] was calculated as described (Ema et al., “Adult mouse hematopoietic stem cells: purification and single-cell assays”, Nat Protoc 2006 1(6), 2979-2987).

Amplex Red Assay

Inhibition capacity of VAP-1 inhibitor szTU73 was measured using Amplex Red assay utilizing Amplex Red reagent (10-acetyl-3,7-dihydroxyphenoxazine; Molecular Probes Europe BV), a highly sensitive and stable probe for H2O2. Fluorescence intensity of the samples was measured (excitation, 545 nm; emission, 590 nm; Tecan ULTRA fluoropolarometer) and H2O2 concentration was calculated from calibration curves generated by serial dilutions of standard H2O2. To evaluate the amount of H2O2 formed via SSAO-mediated reaction by VAP-1 transfected cell lysate, specific enzyme inhibitors, semicarbazide (100 μM) and hydroxylamine (5 μM), were included in the control wells subjected to the same treatments and measurements and these values were subtracted from the total amount of H2O2 formed.

Measurements of ROS Production

Human CD34⁺BM cells were liquid cultured for nine days in StemSpan SFEM medium II (STEMCELL Technologies) containing human stem cell factor (100 ng/ml), FMS-like tyrosine kinase 3 ligand (100 ng/ml), and thrombopoietin (50 ng/ml) (all from Peprotech) with or without LJP-1586. After nine days, the cells were stained with anti-CD38 and anti-CD34 antibodies, washed using DMEM, centrifuged and resuspended in 100 μl DMEM. Then, ROS were detected by DHR-123 reagent (Molecular Probes). For this, DHR-123 was diluted in DMSO and kept as a 5 mM stock solution at −20° C. for single use. The aliquots were thawed, diluted 160 times (30 μM) just before adding 12.5 μl to the HSCs suspended in 100 μl DMEM to a final concentration of 3 μM. The cells were then incubated for 10 min at 37° C. and followed by activation with Phorbol 12-myristate 13-acetate (PMA) (Sigma-Aldrich. The stock solution of PMA was frozen at 1 mg/ml in DMSO, freshly thawed and diluted 500 times in order to add 12.5 μl to a final concentration of 200 ng/ml. After 20 min at 37° C., the cells were washed with PBS, resuspended and analyzed by flow cytometry. The red DHR123 turns to green after oxidation. CD38⁻ and CD34⁺ positive cells were gated and fluorescence intensity of oxidized DHR-123 was measured from the filter channel 530 nm/30 nm using LSR Fortessa instrument (BD Biosciences) and analyzed by FlowJo software (Tree Star).

Colony-Forming Unit (CFU) Assay, Long-Term Culture-Initiating Cell (LTC-IC) Assay, and Liquid Culture

For human umbilical CB cells, an antibody-based EasySep kit was used to enrich CD34⁺CB cells, which were subsequently stained with anti-CD38 and anti-CD34 antibodies. CD38⁻CD34⁺ cells were sorted using a FACSAria Ilu instrument (BD Biosciences) and then cultured in StemSpan SFEM medium II (STEMCELL Technologies) containing human stem cell factor (100 ng/ml), FMS-like tyrosine kinase 3 ligand (100 ng/ml), and thrombopoietin (50 ng/ml) (all from Peprotech). Cells were seeded at a density of 1×103 per ml. LJP-1586 was added immediately after plating when indicated. Cultures were maintained for 21 days, and half the medium was replaced by that containing the same cytokines and LJP-1586 on days 5, 8, 12, 15, and 18.

The progeny of 900 CD38⁻CD34⁺ cells collected from 15-day-old in vitro cultures, obtained as described above, were grown in methylcellulose-based medium (H4436, STEMCELL Technologies) containing or lacking LJP-1586. After 14 days, single, multilineage, and mixed colonies were visually scored by microscopy. Cryopreserved human CD34⁺ cells from AllCells were thawed, resuspended, and counted according to the manufacturer's protocol. Five hundred thawed human BM CD34⁺ cells were cultured in complete methylcellulose-based medium (H4436, STEMCELL Technologies) containning or lacking LJP-1586. The total number of colonies was counted at 14 days after plating. Replating was performed twice by harvesting and dissociating cells under sterile conditions.

Isolation of CD34⁺ Cells and Sorting of VAP-1⁺ and VAP-1⁻HSCs from Human Umbilical CB

CD34⁺ cells from human umbilical CB were isolated via a two-step procedure using Ficoll-Plaque gradient centrifugation (Amersham Pharmacia Biotech, Uppsala, Sweden) and an EasySep Human Cord Blood CD34 Positive Selection Kit II (STEMCELL Technologies). For batch and single cell sorting of VAP-1⁺ and VAP-1⁻ cells from CB we used a Sony SH800 cell sorter with class A2 Level II biosafety cabinet using 130 μm microfluidic sorting chips. This sorter applies low shear stress on cells allowing better survival during cell culture. CD34⁺ cells were also sorted into VAP-1⁺ and VAP-1⁻HSCs (Lineage-CD34+CD38). From these, 100 VAP-1+ and VAP-1−HSCs were then cultured in StemSpan SFEM medium II (STEMCELL Technologies) containing human stem cell factor (100 ng/ml), FMS-like tyrosine kinase 3 ligand (100 ng/ml), and thrombopoietin (50 ng/ml) (all from Peprotech). LJP-1586 was added immediately after plating at a concentration of 1 μM.

Cultures were maintained for 20 days. Fresh medium containing the same cytokines and LJP-1586 was added on days 5, 8, 10, 12, 15 and 18. The cells were analysed on days 10 and 15 for CD38−CD34+CD45RA−CD90+ expression using LSR Fortessa instrument (BD Biosciences). Alternatively, a VAP-1 inhibitor szTU73 was used in CB cultures at concentrations 1, 5 and 10 micromolar.

Results

VAP-1 is Expressed by HSCs and Vascular Endothelial Cells in Human Bone Marrow (BM) and Inhibition of VAP-1 Facilitates their Expansion

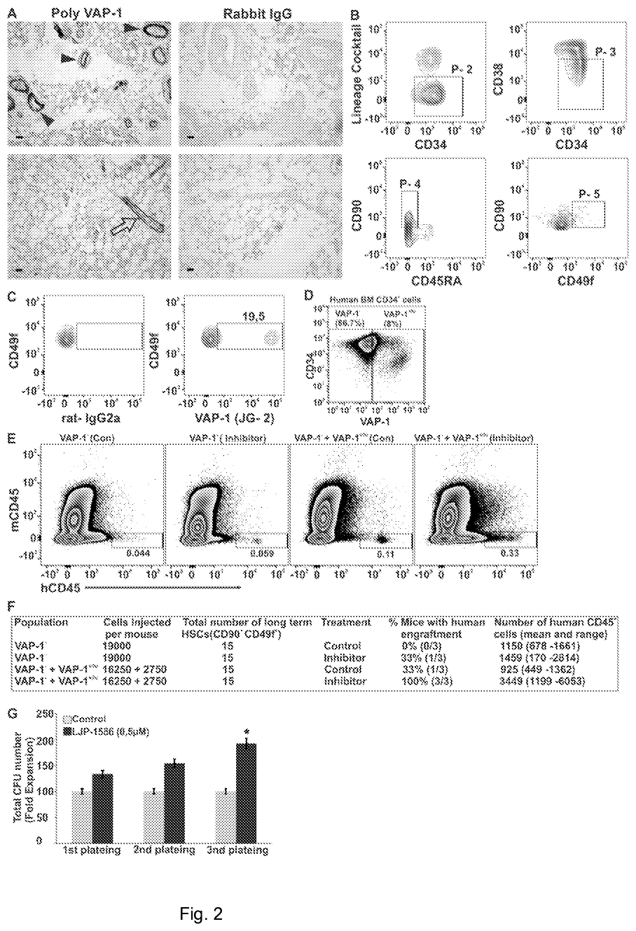

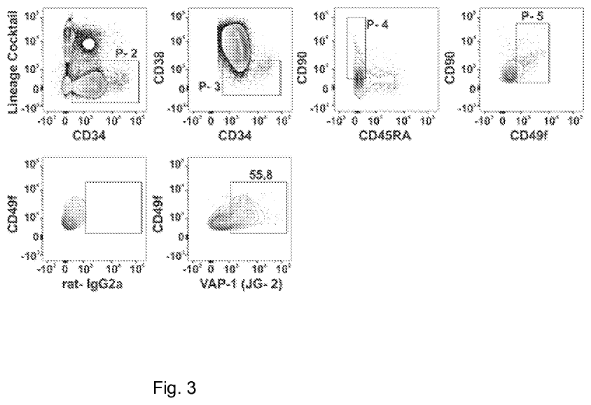

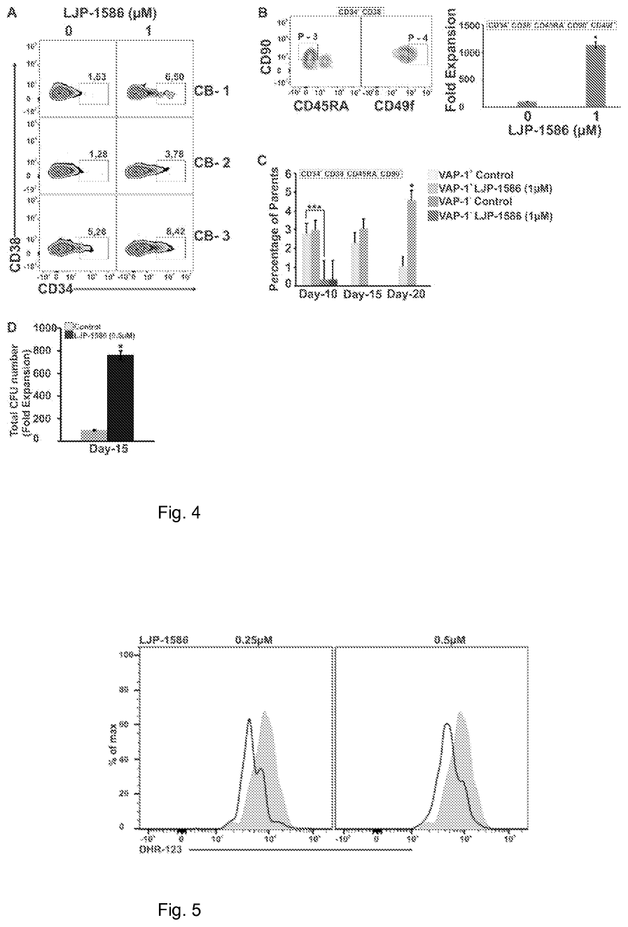

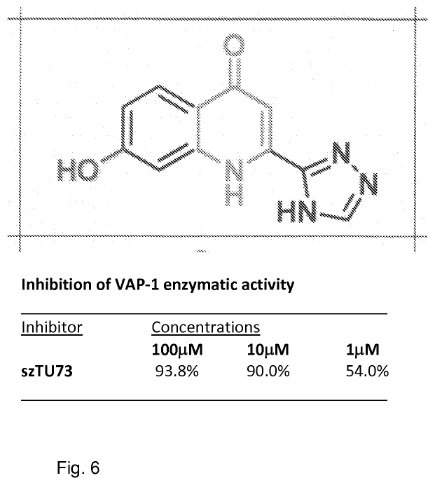

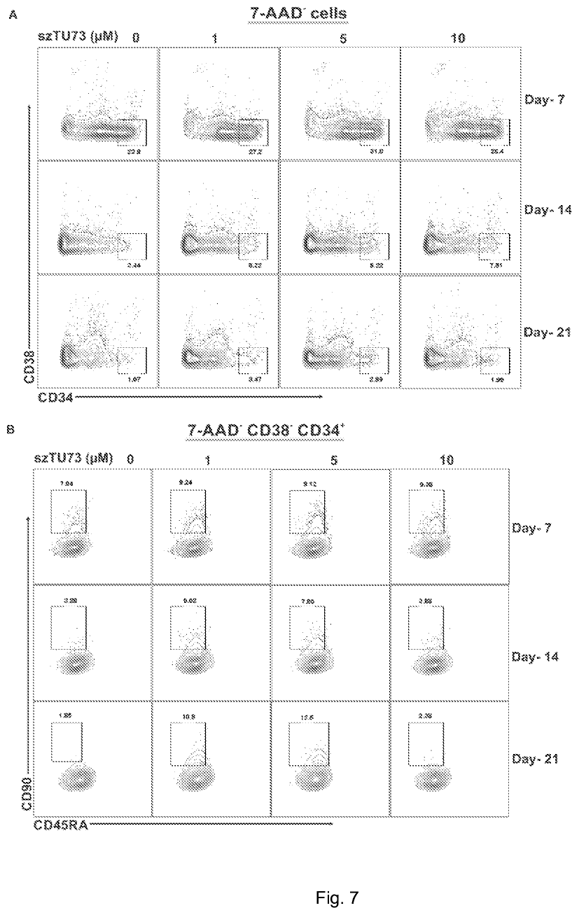

In this Example, we investigated whether human HSCs and blood vascular cells in BM express VAP-1. We detected VAP-1 using a polyclonal anti-VAP-1 antibody in tissue sections of human BM. Arterioles (open arrows) and venules (arrows) were prominently stained by this antibody (FIG. 2A), We studied HSCs in a suspension of CD34⁺ cells prepared from human BM. Flow cytometric analysis of Lineage-CD34⁺CD38⁻CD90⁺CD45RA⁻CD49f⁺ cells among the negative ones revealed that a subset of HSCs expressed VAP-1 on the cell surface as shown in FIGS. 2B and 2C.

We next transferred human VAP-1⁻HSC and a pool containing 14.5% VAP-1⁺ among the negative HSC to NBSGW mice accepting human cells without irradiation and thus, saving the VAP-1 positive BM vasculature intact (FIG. 2D). These mice received either VAP-1 inhibitor or control treatment. Presence of VAP-1⁺ cells in the transfer pool increased the number of CD45⁺ cells (FIG. 2E) of human origin in the BM and 3/3 mice having VAP-1⁺ cells in the transfer pool and receiving the inhibitor accepted the human BM engraftment, whereas none without the VAP-1⁺ cells and inhibitor demonstrated engraftment (FIG. 2F).

To test the function of human HSCs, we performed CFU assays in the presence of LJP-1586. When BM-derived CD34⁺ cells were cultured in methylcellulose-based medium designed for human CFU assays, the number of CFUs formed by LJP-1586-treated cultures was 33% higher than the number of CFUs formed by control cultures. To determine whether these colonies contained HSCs, we dissociated them into single-cell suspensions, re-plated the cells, and repeated this process twice. After this procedure, the number of CFUs formed by LJP-1586-treated cultures was 92% higher than the number of CFUs formed by control cultures (FIG. 2G). These findings demonstrate that BM derived HSCs not only survived but also expanded upon repetitive culture in the presence of LJP-1586.

HSCs in Umbilical Cord Blood (CB) Express VAP-1

Human umbilical CB may be another convenient source of HSCs. CD34⁺ cells isolated from human umbilical CB and analyzed using the HSC markers (FIG. 3), these cells expressed VAP-1. This finding was confirmed using three VAP-1-specific monoclonal antibodies (1B2, TK8-14, and JG-2) which recognize different epitopes of VAP-1. We also confirmed the VAP-1 expression using FACS sorted cord blood CD34⁺ cells. In conclusion, VAP-1 is present on HSCs in umbilical CB.

Inhibition of VAP-1 Facilitates Expansion of Umbilical Cord Blood (CB) Derived Human HSCs In Vitro

Next, we investigated whether inhibition of VAP-1 facilitates the expansion of HSCs in umbilical CB. To this end, we cultured CD34⁺ cells sorted from human CB for 21 days in StemSpan SFEM medium II (Knapp et al., “Dissociation of Survival, Proliferation, and State Control in Human Hematopoietic Stem Cells”, Stem Cell Reports 2017, Jan. 10:8(1), 152-162) containing or lacking various concentrations of LJP-1586 or szTU73, a VAP-1 inhibitor as shown in FIG. 6 using a conventional Amplex Red assay. HSCs expanded more than 31 times in cultures treated with 1 μM LJP-1586 and grown for 18 days compared to the control cells (not containing LJP-1586). Expansion of HSCs was less efficient in cultures treated with higher or lower concentrations of 1 μM LJP-1586. The degree of HSC expansion was donor-dependent but was consistent in samples sorted from a single donor (FIG. 4A). Primitive HSCs were further assessed using the additional markers CD45RA⁻CD90⁺CD49f⁺. More than 12% of HSCs in gate P-3 were primitive HSCs (CD34⁺CD38⁻CD45RA⁻CD90⁺CD49f⁺) and the number of these was 11 times higher in LJP-1586-treated compared to non-treated cultures (FIG. 4B). In conclusion, exposure to LJP-1586 in liquid cultures dramatically expands HSCs (CD34⁺CD38⁻) and primitive HSCs (CD34⁺CD38⁻CD45RA⁻CD90⁺CD49f⁺) compared to the untreated cells. We further tested the capacity of VAP-1− and VAP-1+HSCs to expand in liquid cultures. Unlike in CFU assays, VAP-1+HSCs were the only surviving cell type in long term cultures and the VAP-1 inhibition boosted their expansion on day 20 (FIG. 4C). Similarly, the szTU73-inhibitor was able to expand the hematopoietic stem cells in 21-day cultures, the optimal concentrations being in the range 1-5 micromolar as shown in FIG. 7.

As the inhibitor LJP-1586 blocks the amine oxidase activity of VAP-1, we tested, whether it reduces the concentration of ROS in human HSC cultures and provides them with a growth advantage over non-treated cells. Therefore, we collected the cells and performed oxidative burst assays by using dihydrorhodamine (DHR 123) and flow cytometry. We found that ROS were reduced by 62% (MFI) when the cells were cultured with the LJP-1586 inhibitor compared to the control cells (shown for bone marrow derived HSCs in FIG. 5).

CB-Derived HSCs Expanded in Liquid Cultures in the Presence of LJP-1586 are Fully Functional in Colony Formation

Given that we could expand HSCs obtained from umbilical CB in liquid culture (FIG. 4B, 4C), we investigated the stemness of these cells by the CFU assay. To this end, we collected all cells that had expanded over 15 days in liquid culture in the presence of LJP-1586 and seeded them into methylcellulose-based medium containing LJP-1586. The number of CFUs formed by LJP-1586-treated cultures was 7.9 times higher after 15 days of culture than the number of CFUs formed by control cultures (FIG. 4D). Taken together, these results show that inhibition of VAP-1 facilitates expansion of HSCs in liquid cultures and inhibitor-treated cells are fully capable of forming colonies. Therefore, the method according to the present invention can be used to expand HSCs in clinical settings.

1. A method of producing an expanded population of hematopoietic stem cells ex vivo, said method comprising culturing ex vivo a population of hematopoietic stem cells with a vascular adhesion protein-1 (VAP-1) inhibitor capable of inhibiting the enzymatic activity of vascular adhesion protein-1 (VAP-1), wherein the VAP-1 inhibitor is present in an amount that is sufficient to produce an expanded population of hematopoietic stem cells.

2. The method according to claim 1, wherein said hematopoietic stem cells are human cells.

3. The method according to claim 2, wherein said hematopoietic stem cells are derived from umbilical cord blood, bone marrow and/or peripheral blood.

4. The method according to claim 1, wherein said VAP-1 inhibitor comprises a small molecule inhibitor capable of inhibiting enzymatic activity of VAP-1.

5. A method for controlling reactive oxygen species (ROS) concentration in ex vivo culturing of hematopoietic stem cells, wherein a level of ROS is reduced using vascular adhesion protein-1 (VAP-1) inhibitor to a level providing growth advantage to hematopoietic stem cells.

6. A cell expansion culture medium for hematopoietic stem cells comprising a vascular adhesion protein-1 (VAP-1) inhibitor capable of inhibiting the enzymatic activity of vascular adhesion protein-1 (VAP-1).

7. A method of treating bone marrow suppression or bone barrow failure, wherein the method comprises administering a vascular adhesion protein-1 (VAP-1) inhibitor capable of inhibiting the enzymatic activity of vascular adhesion protein-1 to an individual, wherein said VAP-1 inhibitor maintains and/or expands hematopoietic stem cells (HSC).

8. A method according to claim 6, wherein said VAP-1 inhibitor comprises a small molecule inhibitor capable of inhibiting enzymatic activity of VAP-1.

9. The method according to claim 8, wherein said VAP-1 inhibitor comprises LJP-1586 or szTU73 inhibitor.
